(19) United States
(12) Patent Application Publication
(10) Pub. No.: US 2016/0137571 A1
(43) Pub. Date: May 19, 2016

(54) ALKYL POLY GLYCOL-ETHERS AND THEIR SYNTHESIS

(71) Applicant: Sven Ivar Hommeltoft, Pleasant Hill, CA (US)

(72) Inventor: Sven Ivar Hommeltoft, Pleasant Hill, CA (US)

(73) Assignee: Chevron U.S.A. Inc., San Ramon, CA (US)

(21) Appl. No.: 14/540,618

(22) Filed: Nov. 13, 2014

(51) Int. Cl. C07C 41/09; C07C 45/66; C10M 129/90; C07C 43/11; C10M 129/16; C07C 41/01; C07C 29/145

(52) U.S. Cl. CPC C07C 41/09; C07C 41/01; C07C 45/66; C07C 29/145; C07C 43/11; C10M 129/16; C10M 129/90

(57) ABSTRACT

Processes for producing long chain ethers by reacting a long chain secondary alcohol, a long chain alkoxide, or a combination thereof with at least one epoxide. The long chain ether may comprise a long chain ether alcohol, a long chain ether alcoholate, or a combination thereof. The long chain ether alcoholate may be either protonated to provide the long chain ether alcohol or reacted with an alkyl halide to provide a long chain capped ether. Long chain ether compositions are also disclosed herein.

### TECHNICAL FIELD

This disclosure relates to alkyl poly glycol-ethers and their synthesis.

### BACKGROUND

Some phenyl ethers have in the past been used as lubricants, but these typically have fairly high pour points and are not generally applied.

There is a need for long chain ether lubricants and for processes for efficiently producing long chain ether lubricants.

### SUMMARY

In an embodiment there is provided a process comprising providing a first reactant comprising a material selected from the group consisting of a long chain secondary alcohol, a long chain alkoxide, and combinations thereof; and reacting the first reactant with at least a first epoxide to provide a long chain ether selected from the group consisting of a long chain ether alcoholate of general Formula IIA, a long chain ether alcohol of general Formula IIB, and combinations thereof:

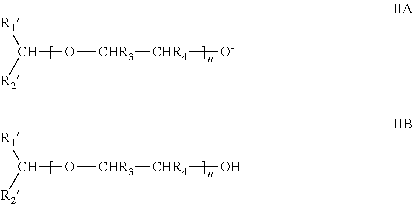

wherein R₁′ and R₂′ are independently selected from the group consisting of C₅-C₂₁ linear or branched alkyl and C₅-C₂₁ linear or branched alkenyl; R₃ and R₄ are independently selected from the group consisting of a hydrogen atom, C₁-C₃₅ linear or branched alkyl, and aryl; and n is an integer from 1 to 50.

In another embodiment there is provided a process comprising contacting at least one fatty acid with a ketonization catalyst in a ketonization zone under ketonization conditions to provide a long chain ketone according to the following Scheme 1:

R₁COOH+R₂COOH→R₁C(O)R₂+CO₂+H₂O

wherein R₁ and R₂ are independently selected from the group consisting of C₅-C₂₁ linear or branched alkyl and C₅-C₂₁ linear or branched alkenyl; and contacting the long chain ketone with a selective ketone hydrogenation catalyst in a ketone hydrogenation zone in the presence of hydrogen gas under selective ketone hydrogenation conditions to provide a long chain secondary alcohol according to the following Scheme 2:

R₁C(O)R₂+H₂→R₁′CH(OH)R₂′

wherein R₁ and R₂ are the same or different, when R₁ is alkyl R₁′═R₁, when R₂ is alkyl R₂′═R₂, when R₁ is alkenyl R₁′ is alkyl or alkenyl, when R₂ is alkenyl R₂′ is alkyl or alkenyl, R₁ and R₁′ have an equal number of carbon atoms, and R₂ and R₂′ have an equal number of carbon atoms. The process further comprises providing a first reactant comprising a material selected from the group consisting of the long chain secondary alcohol, a long chain alkoxide, and combinations thereof; and reacting the first reactant with at least a first epoxide to form a long chain ether selected from the group consisting of a long chain ether alcoholate of general Formula HA, a long chain ether alcohol of general Formula IIB, and combinations thereof:

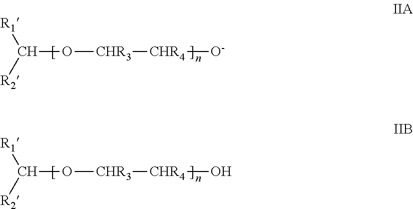

wherein R₁′ and R₂′ are independently selected from the group consisting of C₅-C₂₁ linear or branched alkyl and C₅-C₂₁ linear or branched alkenyl; R₃ and R₄ are independently selected from the group consisting of a hydrogen atom, C₁-C₃₅ linear or branched alkyl, and aryl; and n is an integer from 1 to 50.

In a further embodiment there is provided a long chain ether product prepared by reacting a long chain alkoxide with an epoxide to form a long chain ether alcoholate according to processes as disclosed herein, wherein the long chain ether alcoholate may be reacted with an alkyl halide to provide the long chain ether product. The long chain ether product may be in the range from C₂₆-C₈₆.

In yet another embodiment there is provided a composition comprising at least one long chain ether of general Formula III:

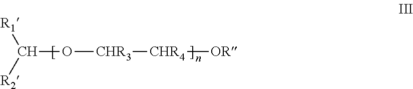

wherein R₁′ and R₂′ are the same or different, and R₁′ and R₂′ are independently selected from the group consisting of C₅-C₂₁ linear or branched alkyl and C₅-C₂₁ linear or branched alkenyl; R₃ and R₄ are the same or different, and R₃ and R₄ are independently selected from a hydrogen atom, C₁-C₃₅ linear or branched alkyl, and aryl; n is an integer from 1 to 50; and R″ is selected from the group consisting of a hydrogen atom, C₁-C₂₂ linear or branched alkyl and C₂-C₂₂ linear or branched alkenyl, with the proviso that when R″ is a hydrogen atom R₁′ and R₂′ jointly contain a total of at least 30 carbon atoms.

### BRIEF DESCRIPTION OF THE DRAWING

FIG. 1 schematically represents an electrolytic cell and process for electrolytically generating an alkoxide from an alcohol, according to an embodiment of the present invention.

### DETAILED DESCRIPTION

Conventional processes for preparing alcohols with a carbon chain length above that of available fatty acids are expensive. Also, such conventional processes place the alcohol group toward the end of the molecule. Also, for lubricant applications it may be important to prepare molecules with a sufficiently high boiling point and viscosity to meet specifications for most lubricant products. This typically requires carbon chains considerably longer than the C₁₆-C₁₈ chains that are made simply by hydrogenation of the most commonly available fatty acids and fatty oil feedstocks.

Applicant has demonstrated a new route to make long chain secondary alcohols, from fatty acids and fatty oils, in which the OH group may be placed non-terminally in the molecule and in which the carbon chain length is about twice (2×) the length of the carbon chain of alcohols prepared by simple hydrogenation of fatty acids and fatty oils. Furthermore, Applicant has discovered that long chain ethers may be prepared from the long chain secondary alcohols and/or their alkoxides via a number of different routes.

Applicant has now discovered that the introduction of an additional oxy-ether linkage in long chain ethers, by reacting a long chain alcohol and/or a long chain alkoxide with an epoxide, may dramatically improve the cold flow properties of the long chain ether products. The deprotonation of the fatty acid-derived long chain alcohol to make the alcoholate anion may be achieved in a plurality of different ways, including reaction of the alcohol with a strong base, reaction of the alcohol with a reactive metal such as sodium, or electrochemically.

The epoxide readily reacts with the alcoholate anions to form ether alcoholate anions. In an embodiment, the reaction of the alcoholate anion with an epoxide may take place in a reaction mixture comprising predominantly a long chain secondary alcohol in combination with lesser amounts of the corresponding alkoxide (alcoholate anion). The reaction of the alcoholate anion with epoxy compounds will typically produce a mixed polyether product in which a variable number of epoxy units have been incorporated. The average number of epoxide molecules incorporated in the epoxide-derived moiety of the long chain ether alcoholate may vary according to the epoxide:(alkoxide+alcohol) molar ratio during the reaction.

The long chain ether alcoholate anions may be reacted with an alkyl halide to make a long chain ether product. Alternatively, the ether alcoholate anions may be protonated (e.g., via hydrolysis) to make an alcohol-ether product with a terminal alcohol group. Such long chain ethers may find applications as lubricants.

### Catalysts for Ketonization

In an embodiment, a suitable catalyst for ketonization may comprise alumina. In an embodiment, the ketonization catalyst may comprise at least 95 wt %, at least 99 wt %, or at least 99.5 wt % alumina. In an embodiment, the fresh ketonization catalyst may be calcined at a temperature in the range from 700 to 1100° F. (371 to 593° C.) for a time period in the range from 0.5 to 24 hours prior to contacting the ketonization catalyst with a reactant (long chain carboxylic acid or fatty acid). In an embodiment, the fresh ketonization catalyst may be calcined in the presence of steam. In an embodiment, the ketonization catalyst may comprise gamma alumina. In an embodiment, the ketonization catalyst may consist essentially of alumina.

In an embodiment, the surface area of the alumina catalyst for ketonization may be in the range from 15 to 500 m²/g of catalyst, or from 50 to 400 m²/g of catalyst, or from 100 to 250 m²/g of catalyst. In an embodiment, an alumina catalyst useful for ketonization reactions as disclosed herein may have various shapes including, for example, granules, pellets, spheres, extrudates, and the like. The alumina catalyst may be disposed within a ketonization zone. A ketonization zone is not limited to any particular reactor type. For example, a ketonization zone may use a fixed-, fluidized-, or moving bed reactor.

Over time, the ketonization catalyst may passivate and lose activity. An alumina catalyst that has become passivated to varying degrees following ketonization may be regenerated, e.g., as described in commonly assigned U.S. patent application Ser. No. ______, filed on even date herewith and entitled Ketonization process using oxidative catalyst regeneration (Atty. Docket No. T-9577).

### Fatty Acid Ketonization

A ketone product may be prepared by contacting at least one fatty acid with a ketonization catalyst in a ketonization zone under ketonization conditions according to the following scheme (Scheme 1), wherein R₁ and R₂ are saturated or unsaturated aliphatic groups, and wherein R₁ and R₂ may be the same or different. As a non-limiting example, R₁ and R₂ may be independently selected from C₅-C₂₁ linear or branched alkyl and C₅-C₂₁ linear or branched alkenyl.

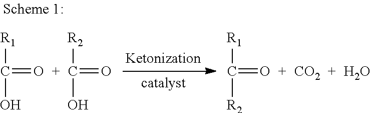

In a sub-embodiment, R₁ and R₂ may be independently selected from C₇-C₁₇ linear or branched alkyl or alkenyl, or from C₉-C₁₇ linear or branched alkyl or alkenyl, or from C₉-C₁₅ linear or branched alkyl or alkenyl, or from C₁₅-C₁₇ linear or branched alkyl or alkenyl. In an embodiment, ketonization may also be known as ketonic decarboxylation or fatty acid decarboxylation-coupling.

In an embodiment, the step of contacting the at least one fatty acid with the ketonization catalyst may comprise feeding a feedstock comprising the at least one fatty acid to the ketonization zone. In an embodiment, feedstocks for ketonization as disclosed herein may be derived from a triglyceride-containing biomass source such as oils or fats from plants and/or animals. In an embodiment, the feedstock may be obtained from biological material (e.g., fatty biomass) having a lipid content greater than (>) 30 wt % on a dry weight basis, or >50, or >70, or >90, or >95, or >99 wt % on a dry weight basis. In an embodiment, the biological material may comprises vegetable oil, animal tallow, algae, and combinations thereof. In an embodiment, the fatty acid feedstock may be derived from other, non-biomass, sources (e.g., Fischer-Tropsch synthesis). Such alternatively derived fatty acids may be mixed or blended with biomass derived fatty acids prior to ketonization, e.g., to alleviate logistical and/or supply related issues involving biomass.

In an embodiment, feedstocks for ketonization may comprise at least one fatty acid reactant or a mixture of fatty acid reactants. In an embodiment, the at least one fatty acid reactant for ketonization may comprise a mixture of at least two (2) fatty acids. In an embodiment, reactants for ketonization may comprise C₆-C₂₂ fatty acids and/or C₆-C₂₂ fatty acid derivatives. In an embodiment, such fatty acid derivatives may include C₆-C₂₂ fatty acid mono-, di-, and triglycerides, C₆-C₂₂ acyl halides, and C₆-C₂₂ salts of fatty acids. In a sub-embodiment, the fatty acids and/or fatty acid derivatives for ketonization may be in the range from C₈-C₁₈, or in the range from C₁₆-C₁₈. In an embodiment, at least one fatty acid for ketonization may be obtained from biological material, including various organisms and biological systems. In an embodiment, the at least one fatty acid may be obtained from at least one naturally occurring triglyceride, for example, wherein the triglyceride may be obtained from biomass. In an embodiment, feedstocks for ketonization may comprise at least 95 wt % fatty acids or at least 99 wt % fatty acids.

In an embodiment, reactants for ketonization may be derived from one or more triglyceride-containing vegetable oils such as, but not limited to, coconut oil, corn oil, linseed oil, olive oil, palm oil, palm kernel oil, rapeseed oil, safflower oil, soybean oil, sunflower oil, and the like. Additional or alternative sources of triglycerides, which can be hydrolyzed to yield fatty acids, include, but are not limited to, algae, animal tallow, and zooplankton.

In an embodiment, reactants for ketonization may include, without limitation, C₈-C₂₂ fatty acids, and combinations thereof. Examples of suitable saturated fatty acids may include, without limitation, caproic acid (C₆), caprylic acid (C₈), capric acid (C₁₀), lauric acid (C₁₂), myristic acid (C₁₄), palmitic acid (C₁₆), stearic acid (C₁₈), eicosanoic acid (C20). Examples of unsaturated fatty acids may include, without limitation, palmitoleic acid, oleic acid, and linoleic acid. Reactants for ketonization may further include, without limitation, palm kernel oil, palm oil, coconut oil, corn oil, soy bean oil, rape seed (canola) oil, poultry fat, beef tallow, and their respective fatty acid constituents, and combinations thereof.

In an embodiment, the reactants for the ketonization reaction or step may be hydrogenated to substantially saturate some or all of the double bonds prior to ketonization. In cases where the fatty oils, i.e. triglycerides, are hydrolyzed to fatty acids, such saturation of the double bonds may be done before or after the hydrolysis.

In some aspects, wherein the above-mentioned hydrolyzed triglyceride sources contain mixtures of saturated fatty acids, mono-unsaturated fatty acids, and polyunsaturated fatty acids, one or more techniques may be employed to isolate, concentrate, or otherwise separate one or more types of fatty acids from one or more other types of fatty acids in the mixture (see, e.g., U.S. Pat. No. 8,097,740 to Miller).

Prior to contacting the reactant with the ketonization catalyst in the ketonization zone, the ketonization catalyst may be calcined. In an embodiment, the step of calcining the ketonization catalyst may be performed in the presence of steam. In an embodiment, the step of calcining the ketonization catalyst may be performed at a temperature in the range from 400 to 600° C., or from 450 to 500° C., for a time period in the range from 0.5 to 10 hours, or from 1 to 2 hours.

In an embodiment, a suitable catalyst for fatty acid ketonization may comprise alumina. In an embodiment, the ketonization catalyst may comprise substantially pure gamma alumina. In an embodiment, the ketonization catalyst may consist essentially of alumina.

Suitable ketonization conditions may include a temperature in the range from 100 to 500° C., or from 300 to 450° C.; a pressure in the range from 0.5 to 100 psi, or from 5 to 30 psi; and a liquid hourly space velocity (LHSV) in the range from 0.1 to 50 h⁻¹, or from 0.5 to 10 h⁻¹. In an embodiment, the partial pressure of the fatty acid in the ketonization zone may be maintained in the range of 0.1 to 30 psi. The ketonization process can be carried out in batch or continuous mode, with recycling of unconsumed starting materials if required.

In an embodiment, the decarboxylation reaction may be conducted in the presence of at least one gaseous- or liquid feedstock diluent. In an embodiment, the ketonization reaction may be carried out while the fatty acid is maintained in the vapor phase. Conditions for fatty acid ketonization are disclosed in commonly assigned U.S. patent application Ser. No. 13/486,097, filed Jun. 1, 2012, entitled Process for producing ketones from fatty acids. In an embodiment, a fatty acid reactant for the ketonization reaction may comprises a mixture of at least two (2) fatty acids such that the ketone product may comprise a mixture of at least three (3) different long chain ketones, each of which may be selectively hydrogenated to provide a mixture of at least three (3) different long chain secondary alcohols.

In an embodiment, the long chain ketones provided by the ketonization reaction can be separated from by-products (such as oligomeric or polymeric species and low molecular weight “fragments” from the fatty acid chains) by distillation. For example, in an embodiment the crude reaction product can be subjected to a distillation-separation at atmospheric or reduced pressure through a packed distillation column. In an embodiment, the ketonization product may be a wax under ambient conditions.

The long chain ketones produced from fatty acids, e.g., as disclosed hereinabove, may be converted to their corresponding long chain secondary alcohol by selective ketone hydrogenation over a selective ketone hydrogenation catalyst, e.g., as disclosed hereinbelow.

### Catalysts for Selective Ketone Hydrogenation

A catalyst for the selective hydrogenation of long chain ketones to the corresponding secondary alcohols may be referred to herein as a “selective ketone hydrogenation catalyst.” In an embodiment, the selective ketone hydrogenation catalyst for selective hydrogenation of long chain (e.g., C₁₁+) ketones may comprise a metal selected from Pt, Pd, Ru, Ni, Co, Mo, Cr, Cu, Rh, and combinations thereof. In an embodiment, the selective ketone hydrogenation catalyst may further comprise a support material. In an embodiment, the support material may be selected from carbon, silica, magnesia, titania, and combinations thereof. In an embodiment, at least some metal component(s) of the hydrogenation catalyst may be in elemental form. As a non-limiting example, the hydrogenation catalyst may comprise a metal selected from Pt, Pd, Ru, Ni, Rh, and combinations thereof, and the metal may be in elemental form in the hydrogenation catalyst. In a sub-embodiment, the hydrogenation catalyst may comprise a metal selected from Pt, Pd, and combinations thereof, and a support material comprising carbon, silica, magnesia, titania, and combinations thereof. In an embodiment, the hydrogenation catalyst may be unsupported meaning, for example, that the metal may be present either in finely divided form (e.g., as metal powder) or in pelletized or extruded or other structural form without the presence of a support material.

In an embodiment, the selective ketone hydrogenation catalyst lacks, or is devoid of, any component that promotes the dehydration of alcohols, such that the hydrogenation catalyst as a whole lacks catalytic activity for dehydration of the long chain secondary alcohol, under the conditions used for the selective hydrogenation of long chain ketones, such that ketone conversion to the corresponding alkene or alkane is prevented. Because the long chain ketones as disclosed herein exhibit comparatively low reactivity in the ketone hydrogenation reaction, e.g., in comparison with C₃ or C₄ ketones, more forcing conditions may be required for hydrogenation as compared to hydrogenation of lighter ketones; such (more forcing) conditions would be expected to exacerbate the negative effect on product selectivity of a hydrogenation catalyst having dehydration functionality. This highlights the significance of using a selective ketone hydrogenation catalyst, in processes as disclosed herein, for the efficient conversion of long chain ketones to the corresponding long chain secondary alcohols in high yield.

In an embodiment, a selective ketone hydrogenation catalyst will lack alumina. As an example, the selective ketone hydrogenation catalyst may be prepared without the use of an alumina component and with a support material, if any, lacking an alumina component, such that the selective ketone hydrogenation catalyst contains at most only trace amounts of alumina that are insufficient to be catalytically effective in dehydrating long chain secondary alcohols under the hydrogenation conditions as disclosed herein for the selective hydrogenation of long chain ketones to the corresponding secondary alcohols.

This is in stark contrast to conventional hydrotreating catalysts having alumina support material that is the major catalyst component by weight and volume. Applicant has observed that the presence of alumina, e.g., in conventional hydrotreating catalysts, negatively impacts the conversion of long chain ketones to the corresponding secondary alcohol product(s) as disclosed herein.

In an embodiment, the surface area of the hydrogenation catalyst may be in the range from 15 to 1000 m²/g of catalyst, or from 100 to 600 m²/g of catalyst, or from 250 to 450 m²/g of catalyst. In an embodiment a selective ketone hydrogenation catalyst, useful for selective hydrogenation of long chain ketones as disclosed herein, may have various shapes including, for example, powder, granules, pellets, spheres, extrudates, and the like. The selective ketone hydrogenation catalyst may be disposed within a ketone hydrogenation zone or ketone hydrogenation reactor. The ketone hydrogenation zone is not limited to any particular reactor type.

### Long Chain Secondary Alcohols by Selective Hydrogenation of Long Chain Ketones

As described hereinabove, a long chain ketone may be prepared, e.g., according to Scheme 1 by contacting at least one fatty acid with a ketonization catalyst in a ketonization zone under ketonization conditions. The long chain ketone may then be selectively hydrogenated by contacting the long chain ketone with a selective ketone hydrogenation catalyst in a ketone hydrogenation zone under selective ketone hydrogenation conditions according to the following Scheme 2 to provide a long chain secondary alcohol.

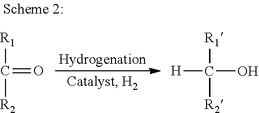

In Schemes 1 and 2, R₁ and R₂ may be the same or different, R₁ and R₂ may be independently selected from C₅-C₂₁ linear or branched alkyl and C₅-C₂₁ linear or branched alkenyl, wherein: when R₁ is alkyl R₁′═R₁, when R₂ is alkyl R₂′═R₂, when R₁ is alkenyl R₁′ is alkyl or alkenyl, when R₂ is alkenyl R₂′ is alkyl or alkenyl, and wherein R₁ and R₁′ have an equal number of carbon atoms, and R₂ and R₂′ have an equal number of carbon atoms. In an embodiment, R₁′ and R₂′ may be independently selected from C₇-C₁₇ linear or branched alkyl, or from C₉-C₁₇ linear or branched alkyl, or from C₉-C₁₅ linear or branched alkyl, or from C₁₅-C₁₇ linear or branched alkyl.

While not being bound by theory, in an embodiment wherein R₁ and R₂ are alkenyl, the product alcohol may be the corresponding saturated alcohol, since alkenyl group hydrogenation is typically more facile than ketone hydrogenation. As an example, when R₁ is alkenyl R₁′ may be alkyl, and when R₂ is alkenyl R₂′ may be alkyl. In a sub-embodiment, R₁′ and R₂′ may be independently selected from the group consisting of C₅-C₂₁ linear or branched alkyl.

In an embodiment, the at least one fatty acid may comprise a mixture of at least two (2) fatty acids, such that the long chain ketone prepared according to Scheme 1 may comprise a mixture of at least three (3) different long chain ketones, and the long chain secondary alcohol prepared according to Scheme 2 may similarly comprise a mixture of at least three (3) different long chain secondary alcohols.

In an embodiment, the selective ketone hydrogenation catalyst will lack catalytic activity for dehydration of the long chain secondary alcohol under the selective ketone hydrogenation conditions used such that, during the step of contacting the long chain ketone with the selective ketone hydrogenation catalyst, ketone conversion to the corresponding alkene or alkane is prevented or hindered. As a result, the corresponding secondary alcohol may be obtained from the long chain ketone with excellent selectivity (e.g., >80% selectivity at 90% conversion).

In an embodiment, a process for preparing long chain secondary alcohols may comprise avoiding contact of the at least one long chain ketone with alumina during the selective ketone hydrogenation step. For example, alumina promotes alcohol dehydration to alkenes, which may in turn be converted to alkanes during conventional hydrogenation, thereby substantially or greatly decreasing the yield of long chain secondary alcohols. Accordingly in an embodiment, the selective ketone hydrogenation catalyst as disclosed herein may be prepared without the use of alumina. In an embodiment, alumina or other material(s) that promote(s) alcohol dehydration may be specifically excluded from the selective ketone hydrogenation catalyst and the ketone hydrogenation zone.

In an embodiment, the selective ketone hydrogenation catalyst may comprise a metal selected from Pt, Pd, Ru, Ni, Co, Mo, Cr, Cu, Rh, and combinations thereof. In an embodiment, the hydrogenation catalyst may further comprise a support material selected from carbon, silica, magnesia, titania, and combinations thereof. In a sub-embodiment, the hydrogenation catalyst may comprise a metal selected from the group consisting of Pt, Pd, and combinations thereof, and a support material selected from carbon, silica, magnesia, titania, and combinations thereof.

In an embodiment, the ketone hydrogenation step may be performed in the absence of a material that promotes dehydration of the long chain secondary alcohol under the selective ketone hydrogenation conditions used, so as to prevent or hinder ketone conversion to the corresponding alkene or alkane, in order to greatly increase the selectivity of ketone conversion to the long chain secondary alcohol product. As a non-limiting example, the selective ketone hydrogenation step may be performed in the absence of alumina. Alumina is used as a catalyst support in conventional hydrotreating catalysts; however, processes as disclosed herein may involve avoiding the presence of alumina during ketone hydrogenation for the production of long chain secondary alcohols. In an embodiment, alumina may be avoided during the ketone hydrogenation step by using a selective ketone hydrogenation catalyst that lacks an alumina component. Selective ketone hydrogenation catalysts that lack alumina are described hereinabove.

In an embodiment, the selectivity of long chain ketone conversion to the corresponding long chain secondary alcohol via the selective ketone hydrogenation step (e.g., according to Scheme 2) may be much higher, e.g., typically at least about 15% higher, than that of comparable ketone hydrogenation in the presence of a conventional hydrotreating catalyst comprising alumina. As a non-limiting example, the selectivity of ketone conversion to the corresponding long chain secondary alcohol by a selective ketone hydrogenation catalyst as disclosed herein may be greater than (>) 80% at 90% conversion, whereas the selectivity of ketone conversion to the corresponding long chain secondary alcohol by a conventional hydrogenation catalyst comprising an alumina support is typically less than (<) 70% at 90% conversion.

In an embodiment, R₁ and R₂ in Schemes 1 and 2 may each be linear or branched alkyl. In a sub-embodiment, R₁ and R₂ may be independently selected from C₅-C₂₁ linear or branched alkyl, or from C₇-C₁₇ linear or branched alkyl, or from C₉-C₁₇ linear or branched alkyl, or from C₉-C₁₅ linear or branched alkyl, or from C₁₅-C₁₇ linear or branched alkyl. In an embodiment, the at least one long chain secondary alcohol formed by ketone hydrogenation, e.g., according to Scheme 2, may be in the range from C₁₁-C₄₃, or from C₂₁-C₃₁, or from C₃₁-C₃₅. In an embodiment, long chain secondary alcohols prepared by processes as disclosed herein may comprise a mixture of long chain secondary alcohols, e.g., each having from 11 to 43 carbon atoms per molecule. In an embodiment, each of the long chain secondary alcohols may have the hydroxyl group placed at a non-terminal location of the molecule. In a further embodiment, a long chain secondary alcohol prepared according to embodiments of processes disclosed herein may have the OH group placed at- or near the center of the secondary alcohol molecule.

In an embodiment, fatty acid ketonization may comprise contacting a mixture of at least two (2) fatty acids with the ketonization catalyst in the ketonization zone. In an embodiment, such a mixture of fatty acids may comprise a lipid mixture derived from a source of lipids selected from a plant, an animal, or other organism(s). Such sources of lipids may include, without limitation, terrestrial plants, mammals, microorganisms, aquatic plants, seaweed, algae, phytoplankton, and the like. In an embodiment, a mixture of fatty acids for ketonization according to processes as disclosed herein may be derived from palm kernel oil, palm oil, coconut oil, corn oil, soy bean oil, rape seed (canola) oil, poultry fat, beef tallow, and the like and their respective fatty acid constituents, and combinations thereof.

In another embodiment, a process for preparing a long chain secondary alcohol may comprise reacting a first fatty acid with a second fatty acid to form a long chain ketone, and selectively hydrogenating the long chain ketone to selectively form the corresponding secondary alcohol.

In an embodiment, the selectively hydrogenating step may comprise contacting the long chain ketone with a selective ketone hydrogenation catalyst in a ketone hydrogenation zone under selective ketone hydrogenation conditions. In an embodiment, the selective ketone hydrogenation catalyst will lack catalytic activity for dehydration of the secondary alcohol, under the selective ketone hydrogenation conditions used, such that ketone conversion to the corresponding alkene or alkane is prevented. Due to the relatively low reactivity of long chain ketones (e.g., C₁₁-C₄₃) in the ketone hydrogenation reaction, as compared with lighter ketones (e.g., C₃ or C₄), the more forcing conditions used for the long chain ketones would exacerbate the negative effect that a hydrogenation catalyst having dehydration functionality would have on product selectivity. Instead, the use of a selective ketone hydrogenation catalyst that at least substantially lacks dehydration activity, as disclosed herein, allows for the efficient conversion of long chain ketones with high selectivity to the corresponding long chain secondary alcohols.

In an embodiment, exemplary conditions for selective ketone hydrogenation may comprise a temperature in the range from 200 to 755° F. (93 to 402° C.), or from 355 to 755° F. (179 to 402° C.), or from 400 to 750° F. (204 to 399° C.), a pressure in the range from 200 to 5000 psi, or from 250 to 5000 psi, or from 300 to 4000 psi, a liquid hourly space velocity (LHSV) in the range from 0.05 to 5.0 h⁻¹, or from 0.1 to 5.0 h⁻¹, or from 0.5 to 4.0 h⁻¹, and a hydrogen to feed molar ratio in the range from 1.0 to 1000, or from 5.0 to 1000, or from 10 to 1000. In an embodiment, the hydrogenation catalyst may comprise a metal selected from the group consisting of Pt, Pd, Ru, Ni, Co, Mo, Cr, Cu, Rh, and combinations thereof. In a sub-embodiment, the metal may be selected from Pt, Pd, and combinations thereof.

As described hereinabove, the selective hydrogenation of long chain ketones may be performed in the absence of a material that promotes dehydration of the secondary alcohol under selective ketone hydrogenation conditions, such that conversion to the corresponding alkene or alkane is prevented or hindered. Accordingly, the selective ketone hydrogenation catalyst will lack a material, such as alumina, that promotes dehydration of the secondary alcohol under said selective ketone hydrogenation conditions. This is in contrast to conventional hydrotreating catalysts having an alumina support that promotes alcohol dehydration to alkenes, with subsequent hydrogenation to alkanes. Advantageously, selective ketone hydrogenation as disclosed herein allows the corresponding secondary alcohol to be obtained efficiently with excellent selectivity.

In an embodiment, long chain secondary alcohol product(s) prepared as disclosed herein may comprise a mixture of long chain secondary alcohols and may be subjected to various separation processes. Such separation may involve, for example, distilling and/or flash distillation to provide one or more long chain secondary alcohol products.

Production of Alkoxides from Alcohols

As noted hereinabove, in an embodiment a long chain ether may be prepared by reacting a long chain alkoxide or an alkoxide/alcohol mixture with one or more epoxides to form ether alcoholate anions or a mixture of ether alcoholate anions and ether alcohols. When the reactant is a long chain alkoxide (alcoholate) the product will be a long chain ether alcoholate; and when the reactant is a mixture of long chain alcohol and long chain alcoholate the product will be a mixture of long chain ether alcohol and long chain ether alcoholate. The long chain ether alcoholate anions may be reacted with an alkyl halide to make an ether capped long chain ether product. The long chain alkoxides may be produced by any suitable method.

In an embodiment, an alcohol may be deprotonated by reaction with a strong base, such as sodium hydride, sodium isopropoxide, or potassium tert-butoxide to directly produce the alkali metal alcoholate from the corresponding alcohol. In another embodiment, an alkoxide may be directly prepared by reacting the alcohol with an alkali metal, such as sodium or potassium. In a further embodiment, an alkoxide may be generated electrolytically from the corresponding alcohol, for example, as described hereinbelow. In another embodiment, a long chain alkoxide may be prepared by reacting a long chain alcohol with a light alkoxide corresponding to a volatile alcohol to form the long chain alkoxide and the volatile alcohol (see, for example, Scheme 4, infra). Alkoxides may also be referred to herein as an alcoholate, an alcoholate salt, or an alcoholate anion.

### Electrolytic Generation of Alkoxides

A long chain alkoxide for producing a long chain ether, may be prepared either: i) directly by feeding a long chain alcohol to a suitable electrolytic cell for conversion of the alcohol to the corresponding long chain alkoxide, or ii) indirectly by reacting a long chain alcohol with an electrolytically generated light alkoxide.

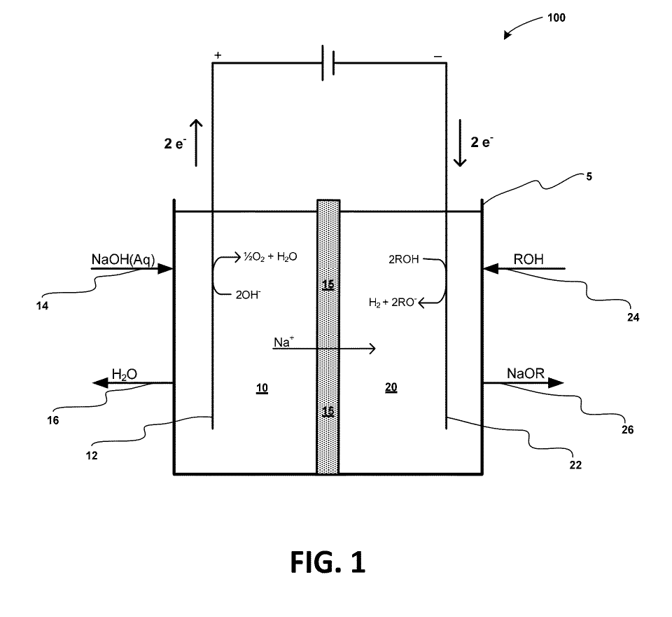

FIG. 1 schematically represents an electrolytic cell and process for electrolytically generating an alkoxide from an alcohol. With further reference to FIG. 1, electrolytic cell 100 may include a vessel 5 housing an anolyte chamber 10 (hereinafter anolyte 10). Electrolytic cell 100 may further include an anode 12 disposed in anolyte 10, an anolyte inlet 14 to anolyte 10, and an anolyte outlet 16 from anolyte 10. Electrolytic cell 100 may further include a catholyte chamber 20 (hereinafter catholyte 20). Electrolytic cell 100 may still further include a cathode 22 disposed in catholyte 20, a catholyte inlet 24 to catholyte 20, and a catholyte outlet 26 from catholyte 20.

Electrolytic cell 100 may still further include a selectively permeable membrane 15. Membrane 15 may be disposed between anolyte 10 and catholyte 20 to define separate anolyte and catholyte chambers, 10 and 20 respectively, of cell 100. In an embodiment, membrane 15 may be an ion conducting membrane. In an embodiment, membrane 15 may be selectively permeable to alkali metal ions, such as Na⁺ ions. In an embodiment, membrane 15 may be at least substantially impermeable to other materials present in anolyte 10 and catholyte 20. Various materials are known in the art to exhibit selective permeability to Na+ or other alkali metal ions. In an embodiment, membrane 15 may comprise a ceramic sodium ion conductor. In an embodiment, membrane 15 may comprise a NaSICON (Na Super Ion CONducting) type material (see, e.g., N. Anantharamulu, et al., J. Mater. Sci. (2011) 46:2821-2837). A non-limiting example of a material for membrane 15 is sodium ionic conductors of the general formula Na₁₊ₓZr₂SiₓP₃₋ₓO₁₂, wherein 0≦x≦3, or (Na₅(Rare Earth)Si₄O₁₂)_{1-δ}((Rare Earth)₂Si₂O₁₀)_{δ}, wherein Rare Earth is Nd, Dy or Sm, and δ is a measure of sodium deficiency in the ceramic membrane.

Liquid(s), e.g., electrolyte solution(s) and the like, may be introduced to and removed from anolyte 10 via anolyte inlet 14 and anolyte outlet 16, respectively. Similarly, electrolytes, substrates, solvents, or the like may be introduced to and removed from catholyte 20 via catholyte inlet 24 and catholyte outlet 26, respectively. Gases, such as hydrogen and oxygen, that may be evolved during operation of cell 100 may be removed from cell 100, e.g., via vents (not shown).

Although the operation of cell 100 will be described primarily with respect to the use of NaOH solution as electrolyte within anolyte 10, it is to be understood that other inorganic sodium salts and other alkali metal salts may also be applicable to the electrolytic generation of various alkoxides. Electrolytic cell 100 and its components are not limited to any particular configuration or materials. Suitable electrically conductive materials that may be used for anode 12 and cathode 22 include materials such as for instance nickel, cobalt, iron, platinum, various alloys, carbon/graphite, and combination thereof.

During operation of cell 100, a solution of NaOH may be introduced into anolyte 10 of electrolytic cell 100 via anolyte inlet 14. Na⁺ ions migrate through membrane 15 from anolyte 10 to catholyte 20 under the influence of an electric potential applied between cathode 22 and anode 12. At the same time, an alcohol substrate (represented as ROH) may be introduced into catholyte 20 via catholyte inlet 24. At the cathode the alcohol reacts with electrons to form hydrogen and alcoholate anions, RO⁻. In catholyte 20, Na+ ions combine with the alcoholate anions to form Na alkoxide, which may be represented generically as NaOR. Within anolyte 10, Off ions are oxidized at anode 12 to produce oxygen and H₂O. In an embodiment, the concentration of Na⁺ ions in anolyte 10 may be maintained in a desired range by introducing fresh NaOH to anolyte 10 via anolyte inlet 14 and by removing diluted NaOH solution from anolyte 10 via anolyte outlet 16. The electrolytically generated sodium alkoxide (NaOR) may be withdrawn from catholyte 20 via catholyte outlet 26. Cell 100 may be operated in continuous mode or batch mode for the electrolytic generation of various alkoxides, including long chain alkoxides and light alkoxides.

Electrolytic generation of an alkoxide from a corresponding alcohol is not limited to any particular type of alcohol substrates. In FIG. 1 and the description thereof, ROH represents an alcohol in a generic sense. As a non-limiting example, the feed, ROH, to catholyte 20 may be a long chain alcohol, a short chain alcohol, a primary alcohol, or a secondary alcohol, and the like. A short chain alcohol may also be referred to herein as a light alcohol or a volatile alcohol.

The electrolytic generation of an alkoxide, represented as MOR, from the corresponding alcohol, ROH, may be represented according to the following Scheme 3:

2MOH+2ROH→2MOR+H₂+½O₂+H₂O

wherein M is an alkali metal.

In an embodiment, the alcohol feed, ROH, to electrolytic cell 100 may comprise a long chain secondary alcohol, wherein the long chain secondary alcohol may be represented as R₁′CH(OH)R₂′. As an example, a long chain alkoxide may be electrolytically generated from the corresponding long chain secondary alcohol according to the following Scheme 3A:

2MOH+2R₁′CH(OH)R₂′→2R₁′CH(OM)R₂′+H₂+½O₂+H₂O

wherein R₁′ and R₂′ are independently selected from C₅-C₂₁ linear or branched alkyl and C₅-C₂₁ linear or branched alkenyl. In an embodiment, such long chain secondary alcohol may be produced from fatty acids, for example, via fatty acid ketonization and selective ketone hydrogenation as described hereinabove (see, e.g., Schemes 1 and 2, supra). In an embodiment, such long chain secondary alcohols may typically comprise from 11 to 43 carbon atoms.

In another embodiment the alcohol feed, ROH, to electrolytic cell 100 may comprise a C₁-C₂₂ primary alcohol. In another embodiment, the alcohol feed, ROH, to electrolytic cell 100 may comprise a volatile alcohol, e.g., in the range from C₁-C₅, to yield a light alkoxide, wherein the light alkoxide may be represented generically as MOR, wherein M represents an alkali metal and R represents a C₁-C₅ linear or branched alkyl group.

In an embodiment, the light alkoxide may be reacted with a long chain alcohol to provide the corresponding long chain alkoxide and the volatile alcohol corresponding to the light alkoxide, e.g., according to the following Scheme 4:

MOR+R₁′CH(OH)R_{2.}′→R₁′CH(OM)R₂′+ROH(g)⇑

wherein MOR represents the light alkoxide, and ROH represents the volatile alcohol, wherein the latter may be evaporated to leave the long chain alkoxide. In an embodiment, R may be selected from a C₁-C₅ linear or branched alkyl group, or a C₂-C₅ linear or branched alkyl group, or a C₃-C₄ linear or branched alkyl group. In a sub-embodiment, R may be selected from isopropyl and tert-butyl.

In a sub-embodiment, the alcohol feed, ROH, to electrolytic cell 100 may comprise a volatile alcohol, such as isopropanol or tert-butanol. As a non-limiting example, an isopropanol feed to electrolytic cell 100 may yield sodium isopropanolate according to the following Scheme 3B:

2NaOH+2iPrOH→2NaOiPr+H₂+½O₂+H₂O

The sodium isopropanolate may then be reacted with a long chain alcohol to provide the corresponding long chain alkoxide and isopropanol, e.g., according to the following Scheme 4A:

NaOiPr+R₁′CH(OH)R₂′→R₁′CH(ONa)R₂′+iPrOH(g)⇑

wherein the isopropanol may be evaporated to yield the long chain alkoxide.

In an embodiment, the alcohol feed, ROH, may be anhydrous or at least substantially anhydrous. In an embodiment, electrolytic cell 100 may be operated at a temperature in the range from ambient temperature to 100° C. Since the melting point of the alkoxides (or alcoholate salts) corresponding to C₁₁-C₄₃ secondary alcohols are typically below (<) 100° C., in an embodiment catholyte 20 may be solvent free, e.g., in an embodiment a substrate feed to catholyte 20 may comprise neat alcohol (ROH). In another embodiment, the alcohol feed, ROH, to catholyte 20 may include a suitable solvent. In an embodiment, the alcohol feed, ROH, to catholyte 20 may comprise a mixture of secondary alcohols. Such a mixture of secondary alcohols may be obtained, e.g., from ketonization of a mixture of two or more fatty acids followed by selective hydrogenation of the resulting mixture of long chain ketones. In an embodiment, a solvent for an alcohol substrate, ROH, being fed to catholyte 20 may comprise one or more lighter alcohols.

In an embodiment, the electrical conductivity of the catholyte solution, e.g., a sodium alkoxide/alcohol mixture, may be increased by the addition to catholyte 20 of a suitable supporting electrolyte. As a non-limiting example, such a supporting electrolyte may comprise a low melting salt, such as a polyalkyl ammonium salt, a pyridinium salt, or an imidazolium salt.

Long Chain Ethers Prepared by Reacting an Alkoxide with an Epoxide

A mixture of long chain secondary alcohols may be prepared from a fatty acid containing feedstock via fatty acid ketonization and selective ketone hydrogenation, as described hereinabove (see, e.g., Schemes 1 and 2). At least a portion of the long chain secondary alcohols may be converted to long chain alkoxides, and the long chain alkoxides and/or long chain alcohols or a mixture thereof may be subsequently reacted with one or more epoxides to provide a mixture of long chain ethers. When the reactant is a mixture of long chain alcohol and long chain alcoholate, the long chain ether product will be a mixture of ether alcohol and ether alcoholate; and when the reactant is a long chain alkoxide (alcoholate) the long chain ether product will be an ether alcoholate. The long chain ether alcoholates may be either: i) ether capped, e.g., by reaction of a long chain ether alcoholate with an alkyl halide, or ii) protonated, e.g., by hydrolysis to provide the ether alcohol. In the case where the ether alcohol is the target product it may only be necessary to have a catalytic amount of the alcoholate present during the epoxide addition reaction involving the long chain alcohol.

In an embodiment, a process for producing a long chain ether may comprise providing a first reactant comprising a material selected from the group consisting of a long chain secondary alcohol, a long chain alkoxide, and combinations thereof. The long chain secondary alcohol may have the following general formula IA:

R₁′CH(OH)R₂′ IA

and the long chain alkoxide may have the following general Formula IB:

R₁′CH(O⁻)R₂′ IB

wherein R₁′ and R₂′ are independently selected from C₅-C₂₁ linear or branched alkyl and C₅-C₂₁ linear or branched alkenyl. The process may further comprise reacting the first reactant with at least a first epoxide to provide a long chain ether. The long chain ether may be selected from a long chain ether alcoholate of general Formula HA, a long chain ether alcohol of general Formula IIB, and combinations thereof:

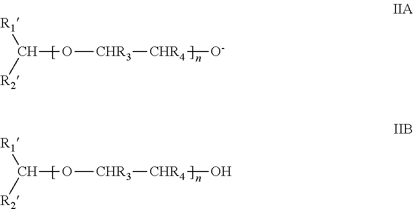

wherein R₁′ and R₂′ are independently selected from C₅-C₂₁ linear or branched alkyl and C₅-C₂₁ linear or branched alkenyl; R₃ and R₄ are independently selected from a hydrogen atom, C₁-C₃₅ linear or branched alkyl, and aryl; and n is an integer from 1 to 50, or from 3 to 30.

In an embodiment, the reaction of a long chain alkoxide with one or more epoxides to provide a long chain ether alcoholate may be represented by the following Scheme 5A:

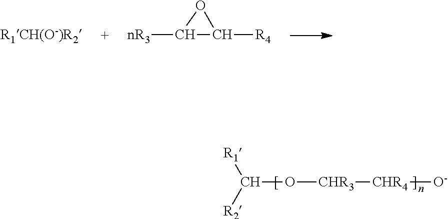

and the reaction of a long chain secondary alcohol with one or more epoxides to provide a long chain ether alcohol may be represented by the following Scheme 5B:

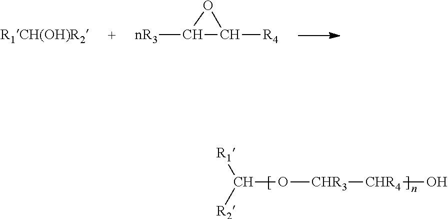

wherein R₁′, R₂′, R₃, R₄, and n are as defined with reference to Formulae IIA and IIB, supra.

With further reference to Schemes 5A and 5B, R₃ and R₄ may be the same or different, and n represents the number of epoxide-derived units incorporated into the long chain ether alcoholate. In an embodiment, n is greater than one such that the long chain ether alcoholate comprises a plurality of epoxide-derived units. In a sub-embodiment, the epoxide-derived units incorporated into the long chain ether alcoholate are the same. In another sub-embodiment, the epoxide-derived units incorporated into the long chain ether alcoholate are different.

In an embodiment, the first reactant (comprising an alkoxide) may be concurrently reacted with the first epoxide and at least a second epoxide, wherein the first epoxide and the second epoxide are different. As an example, the reaction according to Scheme 5A may be conducted using an epoxide mixture comprising a plurality of different epoxide species, such that a plurality of different epoxide-derived units are incorporated into the long chain ether alcoholate, e.g., during a single process step.

In another embodiment, the first reactant (comprising an alkoxide) may be reacted with the first epoxide to provide a first long chain ether alcoholate, and thereafter the first long chain ether alcoholate may be reacted with at least a second epoxide to provide a second long chain ether alcoholate, wherein the first epoxide and the second epoxide are different, and the first long chain ether alcoholate and the second long chain ether alcoholate are different, see, e.g., Scheme 7:

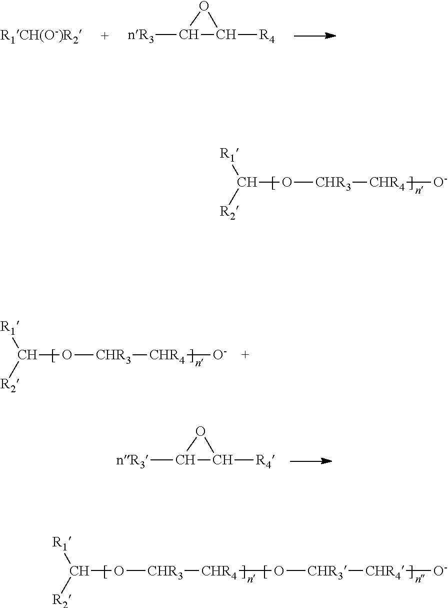

wherein R₁′ and R₂′ are independently selected from the group consisting of C₅-C₂₁ linear or branched alkyl and C₅-C₂₁ linear or branched alkenyl; R₃, R₃′, R₄, and R₄′ are independently selected from the group consisting of a hydrogen atom, C₁-C₃₅ linear or branched alkyl, and aryl, wherein at least one of R₃′ and R₄′ is different from R₃ and R₄; n′ and n″ are integers, and (n′+n″) may be in the range from 2 to 60, or from 3 to 45. In an embodiment, the first long chain ether alcoholate may be sequentially reacted with a series of different epoxide species to successively provide a series of different long chain ether alcoholates.

As a non-limiting illustrative example, an alcoholate prepared according to the reactions depicted in Schemes 5A, 5B, and 6 may involve initial reaction of the R₁CH(O⁻)R₂′ with n′ equivalents of propylene oxide (R₃═H, R₄═CH₃) and then subsequently with n″ equivalents of ethylene oxide (R₃′═R₄′═H) to make an alcoholate of the form R₁′CH(R₂′)—[O—CH₂—CH(CH₃)]_{n′}—[O—CH₂—CH₂]_{n″}—O⁻, wherein n′ and n″ may be the same or different, and (n′+n″) may be in the range from 2 to 60, or from 5 to 50.

In another embodiment, the first reactant may comprise a mixture of a plurality of different long chain secondary alcohol species, and/or a mixture of a plurality of different long chain alkoxide species. In an embodiment, the process may comprise reacting a mixture comprising a plurality of long chain secondary alcohols and/or a plurality of the long chain alkoxides with one or more epoxides to provide a mixture of the long chain ether alcohols and/or a mixture of the long chain ether alcoholates.

With reference to Formulae HA, IIB and III, in a sub-embodiment R₁′ and R₂′ may jointly contain a total of at least 30 carbon atoms. In another sub-embodiment, R₁′ and R₂′ may jointly contain a total number of carbon atoms in the range from 30 to 42. In a further sub-embodiment, R₁′ and R₂′ may be independently selected from C₅-C₂₁ linear or branched alkyl.

In an embodiment, a long chain ether alcoholate may be produced by reacting a long chain alkoxide with at least one epoxide, and a process for producing a long chain ether alcohol product may further comprise protonating the long chain ether alcoholate to provide a compound of the general Formula IIB:

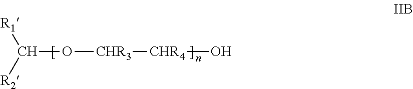

wherein R₁′ and R₂′ may be independently selected from C₅-C₂₁ linear or branched alkyl and C₅-C₂₁ linear or branched alkenyl; n is an integer from 1 to 50; and R₃ and R₄ are independently selected from a hydrogen atom, C₁-C₃₅ linear or branched alkyl, and aryl.

In Formulae IIA and IIB, R₃ and R₄ may be the same or different, and n represents the number of epoxide-derived units incorporated into the long chain ether alcohol/alcoholate. In an embodiment, n is greater than one such that the long chain ether alcohol/alcoholate comprises a plurality of epoxide-derived units, which may be the same or different. As noted hereinabove, a long chain ether alcohol/alcoholate comprising a plurality of different epoxide-derived units may be produced either i) by concurrently reacting a first reactant comprising a long chain alcohol, alkoxide, or mixture thereof with a plurality of different epoxide species, or ii) by sequential reactions involving a plurality of different epoxide species. General Formulae IIA, IIB, and III encompass long chain ether alcoholates, long chain ether alcohols, and long chain ethers prepared by both of the above methods, i) and ii). In an embodiment, a long chain ether product may comprise a mixture of two or more, e.g., a plurality of several or many, compounds of general Formula III.

In an embodiment, a process for producing a long chain capped ether may comprise reacting the long chain ether alcoholate with an alkyl halide to form a long chain ether according to the following Scheme 6:

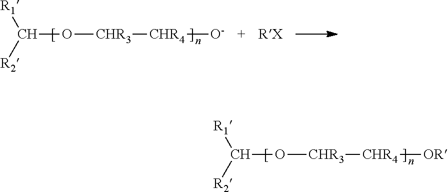

wherein R₁′ and R₂′ may be independently selected from C₅-C₂₁ linear or branched alkyl and C₅-C₂₁ linear or branched alkenyl; n is an integer from 1 to 50; R₃ and R₄ are independently selected from a hydrogen atom, C₁-C₃₅ linear or branched alkyl, and aryl; R′ is selected from C₁-C₂₂ linear or branched alkyl and C₂-C₂₂ linear or branched alkenyl; and X is a halogen atom.

With further reference to Scheme 6, R₃ and R₄ may be the same or different, and n represents the number of epoxide-derived units incorporated into the long chain ether alcoholate. In an embodiment, n may be greater than one such that the long chain ether alcoholate comprises a plurality of epoxide-derived units, wherein the plurality of epoxide-derived units may be the same or different.

According to a further embodiment, there is provided a long chain ether prepared by reacting a long chain ether alcoholate with an alkyl halide, e.g., according to Scheme 6, wherein R₁′ and R₂′ may be independently selected from C₅-C₂₁ linear or branched alkyl and C₅-C₂₁ linear or branched alkenyl. In an embodiment, a long chain ether prepared according to Scheme 6 may be in the range from C₂₆-C₈₆, or from C₃₅-C₈₆, or from C₃₅-C₇₀. In an embodiment, a mixture of long chain ether alcoholates may be reacted with an alkyl halide to provide a long chain ether product comprising a mixture of a plurality of long chain ether compounds.

In an embodiment, a long chain alkoxide having the general Formula IB, supra, may be provided by converting at least a portion of the corresponding long chain secondary alcohol to the long chain alkoxide. The long chain secondary alcohol may be converted to the long chain alkoxide by various methods, for example, as described hereinabove. In an embodiment, the long chain alkoxide from may be electrolytically generated from the long chain secondary alcohol, e.g., as described hereinabove.

In an embodiment, the first reactant may comprise from 0.1 to 99.9 wt % of the long chain secondary alcohol and from 0.1 to 99.9 wt % of the long chain alkoxide. When the first reactant comprises predominantly long chain alkoxide(s), the long chain ether(s) produced by reaction with epoxide(s) may comprise predominantly long chain ether alcoholate(s). When the first reactant comprises predominantly long chain secondary alcohol(s), the long chain ether(s) produced by reaction with epoxide(s) may comprise predominantly long chain ether alcohol(s). In an embodiment, the first reactant may comprise from 50 to 95 wt % of the long chain secondary alcohol and from 5 to 50 wt % of the long chain alkoxide. In another embodiment, the first reactant may comprise from 50 to 95 wt % of the long chain alkoxide and from 5 to 50 wt % of the long chain secondary alcohol. In an embodiment, the first reactant may be provided by converting at least a portion of the long chain secondary alcohol to the corresponding long chain alkoxide.

In an embodiment, the long chain secondary alcohol may in turn be provided by fatty acid ketonization to provide a long chain ketone followed by selective ketone hydrogenation, e.g., as described hereinabove according to Schemes 1 and 2, supra. In an embodiment, fatty acid ketonization may comprise feeding a feedstock comprising at least one fatty acid to a ketonization zone, wherein the feedstock may be obtained from biological material (e.g., fatty biomass) having a lipid content greater than (>) 30 wt % on a dry weight basis, or >50, or >70, or >90, or >95, or >99 wt % on a dry weight basis.

In an embodiment, a given reaction of an alcoholate anion with an epoxy compound will typically produce a mixed polyether product in which a variable number of epoxide-derived units have been incorporated. The average number of epoxide-derived units incorporated into the product may vary according to the molar ratio of epoxide to (alkoxide+alcohol) during the reaction. In an embodiment, the number of epoxide-derived units, n, incorporated into the ether product may have an average value in the range from 2 to 60, or from 3 to 45, or from one (1) to three (3). In an embodiment, the epoxide:(alkoxide+alcohol) molar ratio during the reaction may be in the range from 1 to 50.

As a non-limiting example, the epoxide(s) reacted with the long chain alkoxide/alcohol to provide the ether alcoholate anions may be selected from butene oxide, propylene oxide, and ethylene oxide, or the like, and mixtures thereof. When several different epoxides are used to produce a long chain ether alcoholate they may be reacted either concurrently as a mixture, or consecutively, e.g., by initially adding a number of units of a first epoxide, then a number of units of a second epoxide, and so forth, until a suitable long chain ether alcoholate has been assembled.

The reaction between the first reactant (alcohol/alcoholate feedstock) and the epoxide(s) is typically performed at a temperature at which the reactants and the products form a liquid mixture. Because the melting point of the reactant/product mixture will decrease with conversion, the reaction temperature may also be decreased as the reaction progresses. Typically, the reaction temperature may be in the range from 50-200° C., or from 90-150° C., and the pressure may be from atmospheric to 500 psi, or from atmospheric to 300 psi. In an embodiment, a long chain ether produced according to processes as disclosed herein may be in the range from C₂₆-C₈₆, or from C₃₅-C₈₆, or from C₃₅-C₇₀. In an embodiment, a long chain ether prepared as disclosed herein may have a Viscosity Index greater than (>) 120, or in the range from 120 to 230.

In an embodiment, long chain ether product(s) prepared as disclosed herein, e.g., by reacting an alcoholate anion with an epoxide, may comprise a mixture of long chain ethers and such long chain ether mixtures may be subjected to various fractionation or separation processes. Such separation may involve, for example, distilling and/or flash distillation to provide one or more long chain ether products.

According to another embodiment, there is provided a composition comprising at least one long chain ether of general Formula III:

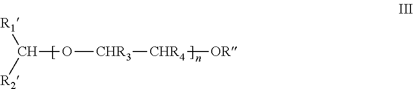

wherein R₁′ and R₂′ are the same or different, and R₁′ and R₂′ are independently selected from C₅-C₂₁ linear or branched alkyl and C₅-C₂₁ linear or branched alkenyl; R₃ and R₄ are the same or different; n is an integer from 1 to 50; and R₃ and R₄ are independently selected from a hydrogen atom, C₁-C₃₅ linear or branched alkyl, and aryl. In Formula III, R″ may be selected from a hydrogen atom, C₁-C₂₂ linear or branched alkyl, and C₂-C₂₂ linear or branched alkenyl, with the proviso that when R″ is a hydrogen atom R₁′ and R₂′ jointly contain a total of at least 30 carbon atoms. In a sub-embodiment, R₁′ and R₂′ may be independently selected from C₅-C₂₁ linear or branched alkyl.

In an embodiment, R₁′ and R₂′ may jointly contain a total number of carbon atoms in the range from 30 to 42. In an embodiment, the number of epoxide-derived units, n, incorporated into a long chain ether product of general Formula III may have an average value in the range from one (1) to three (3). In an embodiment, a long chain ether of general Formula III may be in the range from C₂₆-C₈₆, or from C₃₅-C₈₆, or from C₃₅-C₇₀. In an embodiment, the composition may comprise a mixture of at least two long chain ethers of general Formula III. In an embodiment, a long chain ether of general Formula III may have a Viscosity Index greater than (>) 120, or in the range from 120 to 230.

### Distilling

In an embodiment, a step of distilling may employ one or more distillation columns to separate the desired product(s) from by-products. In an embodiment, the step of distilling may employ flash distillation or partial condensation techniques to remove by-products including at least low molecular weight materials. Those of skill in the art will recognize that there is some flexibility in characterizing the high and low boiling fractions, and that the products may be obtained from “cuts” at various temperature ranges.

### EXAMPLES

### Example 1

### Synthesis of 12-tricosanyl-polybutoxybutyl ether, C₁₁H₂₃CH—O—(CH₂—CHEt-O)ₙ-Bu

40 g (0.117 mole) 12-tricosanol was melted and mixed with 2.92 g (0.127 mole) sodium and reacted at 130° C. for 8 hrs during which time hydrogen gas was evolved and most of the sodium dissolved. Subsequently 17 ml (0.197 mole) 1,2-butene oxide was added and the reaction mixture refluxed at 81° C. with the refluxing butene oxide setting the reflux temperature. As the butene oxide was consumed the temperature was gradually increased up to an externally controlled reaction temperature of 95-100° C. The reaction was continued overnight for about 16 hr at which point 45 ml (0.43 mole) n-butyl chloride was added. The reaction was continued for another 24 hrs during which time a white precipitate (NaCl) formed. Then the product mixture was quenched with 5 g NaHCO₃ in 100 ml water and extracted with 100 ml heptane. The heptane phase was concentrated on a rotary evaporator to 11 torr at 90° C. yielding 58.7 g of an oil with a freezing point well below −20° C. GC composition analysis of this oil showed a mixture of butoxy-butyl ethers with the general formula 12-tricosane-(OBu)n —OBu, n being predominantly 1 and 2 with less than 5% having no epoxide group incorporation (n=0), and less than 20% contained 3 or more epoxide-derived units incorporated. The above oil was dissolved in heptane and cooled to −22° C. at which temperature small amounts of 12-tricosanone and 12-tricosanol precipitated out.

After removal of the solid, the solution was again concentrated at 90° C., 10 torr to yield an oil having the following properties: VI=143, VIS100=3.293, VIS40=12.34 and a melting point below −20° C.

### Example 2

### Coconut Fatty Acid Derived Alkyl-Polypropyleneoxide-Butyl Ether

A secondary alcohol mixture was prepared from coconut fatty acids, generally according to Schemes 1 and 2, supra. Briefly, a mixture of coconut derived, predominantly C₂₁-C₃₁, ketones was prepared by ketonization of a mixture of coconut fatty acids over an alumina catalyst. The mixture of coconut derived ketones was then selectively hydrogenated, en masse, to make a product containing about 90 wt % of a mixture of the corresponding, predominantly C₂₁-C₃₁, secondary alcohols.

Alkyl-polypropyleneoxide-butyl ether was prepared as follows. 102 g of the above mentioned coconut derived secondary alcohol mixture was reacted at 135° C. with 6.1 g (0.26 mole, about 1 equivalent relative to the alcohols) of sodium metal. After 24 hrs almost all of the sodium was dissolved. The temperature was lowered to 90° C. and a slow addition of 120 ml (1.7 mole, about 6.5 equivalents) propylene oxide was started. After 24 hrs all of the propylene oxide had been added and converted. At this point 50 ml (0.48 mole) n-butyl chloride was added and the reaction mixture was left stirring for 2 days. Centrifugation of the product mixture at −10° C. allowed the isolation of 206 g of brownish yellow oil with the following properties: VI=173, VIS100=5.512 cSt, VIS40=24.5 cSt, cloud point=−13° C., pour point=−34° C.

The centrifugation also yielded 31 g of a solid precipitate containing a little additional oil as well as the sodium chloride made in the reaction.

For the purposes of this specification and appended claims, unless otherwise indicated, all numbers expressing quantities, percentages or proportions, and other numerical values used in the specification and claims, are to be understood as being modified in all instances by the term “about.” Furthermore, all ranges disclosed herein are inclusive of the endpoints and are independently combinable. Whenever a numerical range with a lower limit and an upper limit are disclosed, any number falling within the range is also specifically disclosed. Additionally, chemical species including reactants and products designated by a numerical range of carbon atoms include any one or more of, or any combination of, or all of the chemical species within that range.

Any term, abbreviation or shorthand not defined is understood to have the ordinary meaning used by a person skilled in the art at the time the application is filed. The singular forms “a,” “an,” and “the,” include plural references unless expressly and unequivocally limited to one instance. All publications, patents, and patent applications cited in this application are incorporated by reference herein in their entirety to the extent not inconsistent herewith.

Modifications of the exemplary embodiments disclosed above may be apparent to those skilled in the art in light of this disclosure. Accordingly, the invention is to be construed as including all structure and methods that fall within the scope of the appended claims. Unless otherwise specified, the recitation of a genus of elements, materials or other components, from which an individual component or mixture of components can be selected, is intended to include all possible sub-generic combinations of the listed components and mixtures thereof.

It is claimed:

1. A process to make a long chain ether lubricant, comprising:
a) providing a first reactant comprising a material selected from the group consisting of a long chain secondary alcohol, a long chain alkoxide, and combinations thereof; and
b) reacting the first reactant with at least a first epoxide to provide a long chain ether intermediate selected from the group consisting of a long chain ether alcoholate of general Formula IIA, a long chain ether alcohol of general Formula IIB, and combinations thereof: 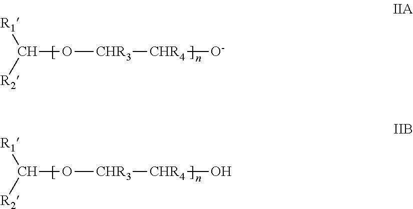
wherein:
R1′ and R2′ are independently selected from the group consisting of C5-C21 linear or branched alkyl and C5-C21 linear or branched alkenyl,
R3 and R4 are independently selected from the group consisting of a hydrogen atom, C1-C35 linear or branched alkyl, and aryl, and
n is an integer from 1 to 50; and
c) preparing the long chain ether lubricant from the long chain ether intermediate;
wherein the long chain ether lubricant has a Viscosity Index from 120 to 230.

2. The process according to claim 1, wherein n represents the number of epoxide-derived units incorporated into the long chain ether intermediate, n is greater than one, and wherein the epoxide-derived units are the same or different.

3. The process according to claim 1, wherein step b) comprises concurrently reacting the first reactant with the first epoxide and with at least a second epoxide, and wherein the first epoxide and the second epoxide are different.

4. The process according to claim 1, wherein the long chain ether intermediate comprises the long chain ether alcoholate, wherein the preparing step c) follows step b), and step c) comprises reacting the long chain ether alcoholate with at least a second epoxide to provide a second long chain ether alcoholate, and wherein the first epoxide and the second epoxide are different.

5. The process according to claim 1, wherein the long chain ether intermediate comprises the long chain ether alcoholate, and the process further comprises:
d) protonating the long chain ether alcoholate to provide the long chain ether alcohol.

6. The process according to claim 1, wherein the long chain ether intermediate comprises the long chain ether alcoholate, and the preparing step c) comprises: reacting the long chain ether alcoholate with an alkyl halide to form the long chain ether lubricant according to the following Scheme 6: 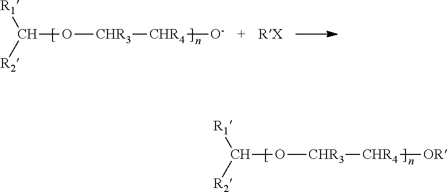
wherein:
R′ is selected from the group consisting of C1-C22 linear or branched alkyl and C2-C22 linear or branched alkenyl, and
X is a halogen atom.

7. The process according to claim 1, wherein
the long chain ether intermediate comprises the long chain ether alcohol.

8. The process according to claim 1, further comprising:
f) contacting at least one fatty acid with a ketonization catalyst in a ketonization zone under ketonization conditions to provide a long chain ketone according to the following Scheme 1:
R₁COOH+R₂COOH→R₁C(O)R₂+CO₂+H₂O
wherein R1 and R2 are independently selected from the group consisting of C5-C21 linear or branched alkyl and C5-C21 linear or branched alkenyl, and
g) contacting the long chain ketone with a selective ketone hydrogenation catalyst in a ketone hydrogenation zone in the presence of hydrogen gas under selective ketone hydrogenation conditions to provide the long chain secondary alcohol according to the following Scheme 2:
C(O)R₂+H₂→R₁′CH(OH)R₂′
wherein:
R1 and R2 are the same or different,
when R1 is alkyl R1′═R1,
when R2 is alkyl R2′═R2,
when R1 is alkenyl R1′ is alkyl or alkenyl,
when R2 is alkenyl R2′ is alkyl or alkenyl,
R1 and R1′ have an equal number of carbon atoms, and
R2 and R2′ have an equal number of carbon atoms; and
h) converting at least a portion of the long chain secondary alcohol to the long chain alkoxide.

9. The process according to claim 1, wherein the first reactant comprises from 0.1 to 99.9 wt % of the long chain secondary alcohol and from 0.1 to 99.9 wt % of the long chain alkoxide.

10. The process according to claim 1, wherein the first reactant comprises from 50 to 95 wt % of the long chain secondary alcohol and from 5 to 50 wt % of the long chain alkoxide.

11. The long chain ether lubricant prepared according to the process of claim 6, wherein the long chain ether lubricant is in the range from C26-C86.

12. A process to make a long chain ether lubricant, comprising:
a) contacting at least one fatty acid with a ketonization catalyst in a ketonization zone under ketonization conditions to provide a long chain ketone according to the following Scheme 1:
R₁COOH+R₂COOH→R₁C(O)R₂+CO₂+H₂O
wherein R1 and R2 are independently selected from the group consisting of C5-C21 linear or branched alkyl and C5-C21 linear or branched alkenyl;
b) contacting the long chain ketone with a selective ketone hydrogenation catalyst in a ketone hydrogenation zone in the presence of hydrogen gas under selective ketone hydrogenation conditions to provide a long chain secondary alcohol according to the following Scheme 2:
R₁C(O)R₂+H₂→R₁′CH(OH)R₂′
wherein:
R1 and R2 are the same or different,
when R1 is alkyl R1′═R1,
when R2 is alkyl R2′═R2,
when R1 is alkenyl R1′ is alkyl or alkenyl,
when R2 is alkenyl R2′ is alkyl or alkenyl,
R1 and R1′ have an equal number of carbon atoms, and
R2 and R2′ have an equal number of carbon atoms;
c) providing a first reactant comprising a material selected from the group consisting of the long chain secondary alcohol, a long chain alkoxide, and combinations thereof;
d) reacting the first reactant with at least a first epoxide to form a long chain ether intermediate selected from the group consisting of a long chain ether alcoholate of general Formula IIA, a long chain ether alcohol of general Formula IIB, and combinations thereof: 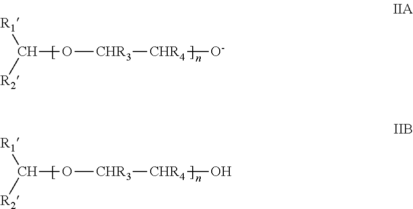
wherein:
R1′ and R2′ are independently selected from the group consisting of C5-C21 linear or branched alkyl and C5-C21 linear or branched alkenyl,
R3 and R4 are independently selected from the group consisting of a hydrogen atom, C1-C35 linear or branched alkyl, and aryl,
n is an integer from 1 to 50; and
e) preparing the long chain ether lubricant from the long chain ether intermediate;
wherein the long chain ether lubricant has a Viscosity Index from 120 to 230.

13. The process according to claim 12, wherein n represents the number of epoxide-derived units incorporated into the long chain ether intermediate, n is greater than one, and wherein the epoxide-derived units are the same or different.

14. The process according to claim 12, wherein step d) comprises concurrently reacting the first reactant with the first epoxide and with at least a second epoxide, and wherein the first epoxide and the second epoxide are different.

15. The process according to claim 12, wherein the long chain ether intermediate comprises the long chain ether alcoholate, and the process further comprises: between step d) and e), reacting the long chain ether alcoholate with at least a second epoxide to provide a second long chain ether alcoholate, wherein the first epoxide and the second epoxide are different.

16. The process according to claim 12, wherein the long chain ether intermediate comprises the long chain ether alcoholate, and the preparing step e) comprises reacting the long chain ether alcoholate with an alkyl halide to form the long chain ether lubricant according to the following Scheme 6: 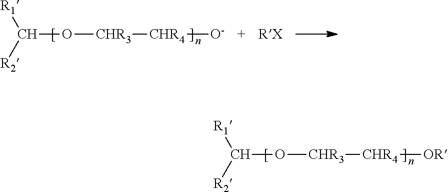
wherein:
R′ is selected from the group consisting of C1-C22 linear or branched alkyl and C2-C22 linear or branched alkenyl, and
X is a halogen atom.

17. (canceled)

18. (canceled)

19. (canceled)

20. (canceled)

21. The process of claim 1, wherein the reacting step b) improves a cold flow property of the long chain ether lubricant.

22. The process of claim 12, wherein the reacting step d) improves a cold flow property of the long chain ether lubricant.

23. A process to make a long chain ether lubricant, comprising:
a) contacting at least one fatty acid with a ketonization catalyst in a ketonization zone under ketonization conditions to provide a long chain ketone according to the following Scheme 1:
R₁COOH+R₂COOH→R₁C(O)R₂+CO₂+H₂O
wherein R1 and R2 are independently selected from the group consisting of C5-C21 linear or branched alkyl and C5-C21 linear or branched alkenyl, and
b) contacting the long chain ketone with a selective ketone hydrogenation catalyst in a ketone hydrogenation zone in the presence of hydrogen gas under selective ketone hydrogenation conditions to provide the long chain secondary alcohol according to the following Scheme 2:
R₁C(O)R₂+H₂→R₁′CH(OH)R₂′
wherein:
R1 and R2 are the same or different,
when R1 is alkyl R1′═R1,
when R2 is alkyl R2′═R2,
when R1 is alkenyl R1′ is alkyl or alkenyl,
when R2 is alkenyl R2′ is alkyl or alkenyl,
R1 and R1′ have an equal number of carbon atoms, and
R2 and R2′ have an equal number of carbon atoms; and
c) converting at least a portion of the long chain secondary alcohol to a long chain alkoxide;
d) reacting the long chain alkoxide with at least a first epoxide to provide a long chain ether intermediate consisting of a long chain ether alcoholate of general Formula IIA: 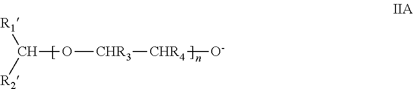
e) reacting the long chain ether alcoholate with an alkyl halide to form the long chain ether lubricant according to the following Scheme 6: 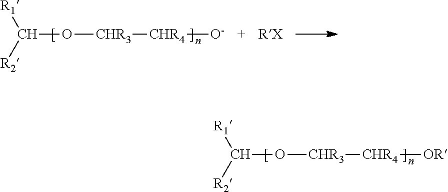
wherein:
R′ is selected from the group consisting of C1-C22 linear or branched alkyl and C2-C22 linear or branched alkenyl, X is a halogen atom, R1′ and R2′ are independently selected from the group consisting of C5-C21 linear or branched alkyl and C5-C21 linear or branched alkenyl,
R3 and R4 are independently selected from the group consisting of a hydrogen atom, C1-C35 linear or branched alkyl, and aryl, and
n is an integer from 1 to 50; and
wherein the long chain ether lubricant has a Viscosity Index from 120 to 230.
